(19) United States
(12) Patent Application Publication
(10) Pub. No.: US 2003/0170265 A1
(43) Pub. Date: Sep. 11, 2003

(54) Use of grifola frondosa fungus extracts

(76) Inventors: Florence Henry, Villers-Les-Nancy (FR); Louis Danoux, Sauixures-Les-Nancy (FR); Gilles Pauly, Nancy (FR)

Correspondence Address: COGNIS CORPORATION, 2500 RENAISSANCE BLVD., SUITE 200, GULPH MILLS, PA, 19406

(21) Appl. No.: 10/332,177

(22) PCT Filed: Jun. 27, 2001
(86) PCT No.: PCT/EP01/07335

(30) Foreign Application Priority Data
Jul. 6, 2000 (EP) 004402095

(51) Int. Cl. A61K 7/42; A61K 35/84

(52) U.S. Cl. 424/195.15; 424/59

(57) ABSTRACT

Methods for treating skin by contacting the skin with an extract of Grifola frondosa are described, along with skin-care and hair-care compositions containing such extracts and processes for preparing such extracts.

### FIELD OF THE INVENTION

This invention relates generally to cosmetic and dermatological care preparations and more particularly to the use of extracts of edible fungi for a variety of applications in cosmetic and dermatological care preparations.

### PRIOR ART

It is known that extracts of higher edible fungi, the so-called Basidiomycetes, have significant therapeutic potential in medicine. Thus, extracts of edible fungi are used in the treatment of AIDS and in anti-tumor therapy. Such extracts have also been successfully used in the treatment of diabetes and obesity. The above-mentioned edible fungi from the class Basidiomycetes and the order Agaricales (agarics) are, for example, Agaricus campester (field mushroom), Agaricus blazei, Panellus serotinus, Grifola frondosa (maitake) or Tremella fuciformis.

Some cosmetic preparations contain a combination of extracts of numerous individual higher fungi and are used, for example, as skin whiteners (JP 02049710) or as anti-allergics (JP 1228480). However, it is not clearly explained in these documents which of the fungal extracts is responsible for the particular effect and which extract represents the essential ingredient of the cosmetic preparation for this type of cosmetic application.

Today, cosmetic preparations are available to the consumer in a variety of combinations. Nevertheless, there is a need on the market for products with an improved performance spectrum. Dematological compatibility and the use of natural products are demanded by consumers. Manufacturers of products suitable for a variety of applications have hitherto been confronted by the problem of having to add to their preparations a large number of active principles which, together, provide the required performance profile without affecting one another or without producing unwanted side effects. Accordingly, there is a particular interest in care preparations which combine the desired properties. In addition, it is desirable to obtain distinctly better products by discovering new applications for existing classes of compound. Extracts of renewable raw materials in particular and their ingredients are being used increasingly more frequently in the field of cosmetics.

### DESCRIPTION OF THE INVENTION

The problem addresed by the present invention was to provide extracts of renewable raw materials for cosmetic and/or dermatological application which would be available in large quantities and which could be widely used as care preparations in various areas of cosmetology and/or dermatology.

Another problem addressed by the invention was to provide extracts of higher fungi as renewable raw materials which could be used in cosmetology and/or dermatology.

The present invention relates to the use of extracts of Grifola frondosa in cosmetic and/or dermatological skin-care preparations.

It has surprisingly been found that, by using extracts of Grifola frondosa, it is possible to obtain care preparations which combine a number of favorable skin care and skin protection properties and which, in addition, show high dermatological compatibility.

The multiple applications according to the invention of the extract of the renewable raw material Grifola frondosa make it very attractive both to the market and to the consumer. Accordingly, the complex problem addressed by the invention has been solved by the use of extracts of Grifola frondosa.

Renewable raw materials in the context of the present invention are understood to be both whole higher fungi and parts thereof (receptacle, stalk, mycelium) and mixtures thereof. According to the invention, the receptacles are particularly preferred for extracting the fungus.

Grifola frondosa

The extracts to be used in accordance with the invention are obtained from higher fungi of the class Basidiomycetes and, more particularly, are extracts of the edible fungus Grifola frondosa which is also known as maitake. This fungus belongs to the so-called bracket fungi which are also known as basidium fungi. These fungi have the major advantage that they can be cultivated in large quantities. Availability is very high and unaffected by the seasons.

Extraction

The extracts to be used in accordance with the invention may be prepared by typical methods of extraction. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found, for example, in Hagers Handbuch der pharmazeutischen Praxis (5th Edition, Vol. 2, pp. 1026-1030, Springer Verlag, Berlin-Heidelberg-New York 1991). Fresh or dried fungi or parts thereof are suitable as the starting material although fungi and/or parts thereof which may be mechanically size-reduced before extraction are normally used. Any size reduction methods known to the expert, for example crushing in a mortar, may be used.

Preferred solvents for the extraction process are water, organic solvents or mixtures of organic solvents and water, more particularly low molecular weight alcohols, hydrocarbons, ketones, esters or halogenated hydrocarbons with more or less large water contents (distilled or non-distilled), preferably aqueous alcoholic solutions with a temperature of 20° C. or higher. Extraction with water, methanol, ethanol, hexane, cyclohexane, pentane, acetone, propylene glycols, polyethylene glycols, ethyl acetate, dichloromethane, trichloromethane and mixtures thereof is particularly preferred. The extraction process is generally carried out at 20 to 100° C. and preferably at 20 to 85° C., more particularly either at the boiling temperature of the solvent used or at room temperature. In one possible embodiment, the extraction process is carried out in an inert gas atmosphere to avoid oxidation of the ingredients of the extract. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of dried fungi or dried parts of fungi (optionally degreased) are in the range from 1.5 to 25 and preferably 1.9 to 20.3% by weight. The present invention includes the observation that the extraction conditions and the yields of the final extracts may be selected according to the desired application. If desired, the extracts may then be subjected, for example, to spray drying or freeze drying.

The quantity of fungal extracts used in the preparations mentioned is governed by the concentration of the individual ingredients and by the way in which the extracts are used. In general, the total quantity of fungal extract present in the preparations according to the invention is 0.001 to 25% by weight, preferably 0.03 to 5% by weight and more particularly 0.03 to 0.1% by weight, based on the final preparation, with the proviso that the quantities add up to 100% by weight with water and optionally other auxiliaries and additives.

The total content of auxiliaries and additives may be 1 to 50% by weight and is preferably 5 to 40% by weight, based on the final cosmetic and/or pharmaceutical preparation. The preparations may be produced by standard cold or hot processes but are preferably produced by the phase inversion temperature method.

In the context of the present invention, the terms “preparations”, “final preparations” and “compositions” may be regarded as synonymous with the term “care preparations”.

Active substance in the context of the invention relates to the percentage content of substances and auxiliaries and additives which are present in the preparations except for the water additionally introduced.

Care Preparations

Care preparations in the context of the invention are understood to be skin care preparations. These care preparations possess inter alia stimulating, healing and restorative effects on the skin. Preferred care preparations in the context of the invention are those which have a stimulating effect on the skin cells and their functions and, in addition, show a restorative effect on the skin and a protective effect against environmental influences on the skin. Other preferred care preparations in the context of the invention are those which are capable of either improving or healing various skin diseases with their different effects on the appearance and function of the skin.

The preparations according to the invention have an excellent skin care effect coupled with high dermatological compatibility. In addition, they show high stability, particularly towards oxidative decomposition of the products.

The present invention also relates to the use of extracts of the fungus Grifola frondosa in protective and restorative care preparations for stimulating the metabolism of the skin cells with revitalizing and reactivating activity on the skin. The strengthening of the natural functions of the skin is effected above all by stimulation of the metabolism of the body's own skin cells through the use of the Grifola frondosa extract.

The present invention also relates to the use of extracts of the fungus Grifola frondosa in care preparations for stimulating the immune defense of the skin. This way of using the care preparations has a positive effect, for example against the negative influence of environmental pollution on the skin, by reactivating the natural functions of the skin and making the skin more resistant and hence strengthening the immune defense of the skin.

The present invention also relates to the use of extracts of the fungus Grifola frondosa in cosmetic and/or dermatological care preparations for stimulating the synthesis of dermal macromolecules selected from the group consisting of collagen, elastin, fibronectin, proteoglycans and hyaluronic acid and salts thereof.

Dermal Macromolecules

Dermal macromolecules in the context of the invention are, in principle, any macromolecules which can be found as constituents of the skin either in the basal membrane between the dermis and the epidermis or directly in the dermis and epidermis. More particularly, they are compounds selected from the group consisting of collagen, elastin, proteoglycans, fibronectins and hyaluronic acid and salts thereof. Collagen consists of protein fibers and occurs in human skin in three different types (type I, III and IV). In collagen, the individual polypeptide chains—which contain much of the amino acid proline and, as every third residue, glycine—are wound around one another to form a triple helix. The collagen fibers are synthesized as tropocollagen in the fibroblasts and are displaced into the extracellular matrix. The stimulation of the collagen synthesis in accordance with the invention leads to an increase in the production of collagen and hence to increased intermolecular stiffening of the dermis and thus to firmer looking skin. Elastin is also a fibrous protein. It consists of unstructured, covalently crosslinked polypeptide chains which form a rubber-like elastic material. After synthesis in the skin cells, the elastin is displaced into the extracellular matrix. The stimulation of the synthesis of the elastin polypeptide chains in accordance with the invention leads to an increase in the production of elastin and hence to an increase in the elasticity of the skin.

Like the glycoproteins, the proteoglycans consist of carbohydrates and proteins. With the proteoglycans, however, the percentage content of polysaccharides is predominant. The proteoglycans of the skin contain dermatan sulfate. Around 140 such proteoglycans are non-covalently attached by small proteins (link proteins) to a hyaluronic acid chain to form molecular aggregates with an average molecular weight of ca. 2 million. The polyanionic aggregates, which are distinguished by their water binding capacity, are capable of forming solid gels which provide the supporting tissue (extracellular matrix) with elasticity and tensile strength. In mucosa, they protect the epithelia. The stimulation of the synthesis of proteoglycans and hyaluronic acid in accordance with the invention leads to a larger quantity of extracellular matrix and hence to greater elasticity and tensile strength.

Fibronectin represents a group of high molecular weight glycoproteins (MW of the dimer ca. 440,000-550,000) which are found in the extracellular matrix and in extracellular fluids. By linear combination of three different, recurring domains, the fibronectin dimer (an elongate molecule measuring 600×25 A) which is joined by two disulfide bridges binds inter alia collagens, glycosaminoglycans, proteoglycans, fibrin(ogen), deoxyribonucleic acids, immunoglobulins, plasminogen, plasminogen activator, thrombospondin, cells and microorganisms. These properties enable it, for example, to bind connective tissue cells to collagen fibrils or thrombocytes and fibroblasts to fibrin (contribution to wound healing).

Hyaluronic acid is an acidic glycosaminoglycan. The basic unit of hyaluronic acid is an aminodisaccharide which is produced from D-glucuronic acid and N-acetyl-D-glucosamine in (beta 1-3) glycosidic linkage and which is attached to the next unit by a (beta 1-4) glycosidic bond.

In addition, the extracts according to the invention may be used in cosmetic and/or dermatological care preparations for reducing the proteolysis and glycation of macromolecules in the skin. Proteolysis is a process in which proteins are split by hydrolysis of the peptide bonds by acids or enzymes. Another name is proteinase digestion. The reduction in proteolysis in accordance with the invention leads to reduced cleavage of the dermal macromolecules with a protein structure and hence to prevention of any reduction in strengthening of the skin and to prevention of any decline in an increased elasticity. Glycation is a non-enzymatic reaction of glucose or other sugars with proteins to form glycoproteins. This reaction results in unintended modifications to the collagen and elastin and hence in changes to the extracellular matrix. The function of the collagen and the extracellular matrix is disrupted. The prevention of glycation in accordance with the invention leads to a reduction in the non-enzymatic modification of collagen and elastin and hence to prevention of a reduced function of the extracellular matrix.

The present invention relates to the use of extracts of Grifola frondosa in care preparations for the preventive or healing treatment of signs of skin ageing. Another name for care preparations of this type is anti-ageing preparations. These signs of ageing include, for example, any type of wrinkling and lining. The treatments include the retardation of skin ageing processes. The ageing signs can have various causes. Above all, they are caused by UV-induced skin damage. In one particular embodiment of the invention, the care preparations are used for the treatment of UV-induced ageing of the skin.

The present invention also relates to the use of extracts of the fungus Grifola frondosa as a cosmetic and/or dermatological anti-inflammatory care preparation. Anti-inflammatory care preparations in the context of the invention are care preparations which are capable of healing or preventing inflammation of the skin. The inflammation can have various causes. More particularly, inflammation induced by UV radiation, skin contamination or bacterially or hormonally induced changes in the skin, for example acne, can be treated. Test results have shown that the extracts according to the invention with strong anti-inflammatory effects contain little or no fumaric acid. Accordingly, the anti-inflammatory effect cannot be attributed to the presence of fumaric acid.

The present invention also relates to the use of extracts of the fungus Grifola frondosa in cosmetic and/or dermatological care preparations for sensitive skin.

The present invention also relates to the use of extracts of the fungus Grifola frondosa is cosmetic and/or dermatological care preparations for improving wound healing. The improvement of wound healing in the context of the invention is understood to encompass improvements which are capable of supporting, stimulating and strengthening the natural healing process in the event of disease-induced changes to the skin. The disease-induced changes to the skin can have various causes, one possible cause being injury.

The present invention also relates to the use of extracts of the fungus Grifola frondosa in cosmetic and/or dermatological sun protection compositions.

Sun protection compositions or UV protection factors in the context of the present invention are compositions which are useful in protecting the human skin against the harmful effects of direct and indirect solar radiation. The UV radiation of the sun which is responsible for tanning the skin is divided into the sections UV-C (wavelengths 200-280 nm), UV-B (280-315 nm) and UV-A (315-400 nm).

The pigmentation of normal skin under the influence of solar radiation, i.e. the formation of melanins, is differently produced by UV-B and UV-A. Exposure to UV-A rays (“long-wave UV”) results in darkening of the melanins already present in the epidermis without any sign of harmful effects. The situation is different with so-called “short-wave UV” (UV-B). This leads to the formation of so-called late pigment through the re-formation of melanins. However, before the (protective) pigment is formed, the skin is exposed to the effect of the unfiltered radiation which can lead to reddening of the skin (erythemas), inflammation of the skin (sunburn) and even blisters, depending on the exposure time.

The extracts of the fungus Grifola frondosa according to the invention are used as UV absorbers or filters, which convert the UV radiation into harmless heat, and in addition may be used in combination with other sun protection compositions or UV protection factors.

These other UV protection factors are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)-camphor as described in EP 0693471 B1;

4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably 4-methoxybenzalmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB);

propane-1,3-diones such as, for example, 1-(4-tert.butylphenyl)-3-(4′-methoxyphenyl)-propane-1,3-dione;

ketotricyclo(5.2.1.0)decane derivatives as described in EP 0694521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 19712033 A1 (BASF). The UV-A and UV-B filters may of course also be used in the form of mixtures. Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used. Other suitable UV filters can be found in P. Finkel's review in SÖFW-Journal 122, 543 (1996) and in Parf\ddot{m}erie und Kosmetik 3 (1999), pages 11 et seq.

The extracts according to the invention may also be used in cosmetic and/or dermatological care preparations as tyrosinase inhibitors and/or as skin whiteners. Skin whiteners make the skin lighter in appearance. One way of lightening or whitening the skin involves the inhibition of tyrosinase because tyrosinase is involved in the formation of the skin pigment melanin (depigmentation). The use of extracts of Grifola frondosa in accordance with the invention leads through tyrosinase inhibition to reduced formation of melanin and hence to whitening of the skin. The extracts of Grifola frondosa may additionally be used in combination with other tyrosinase inhibitors, for example arbutin, ferulic acid, koji acid, coumaric acid and ascorbic acid (vitamin C), as depigmenting agents.

The use of the extracts in accordance with the invention as protective and restorative care preparations is possible in principle for any preparations which are used to prevent damage or in cases of skin damage and hence in skin care. Another use in this field is application in people with sensitive skin damaged by allergies or other causes. The skin damage may be caused by various factors.

In principle, the extracts according to the invention may be used in any cosmetic products. Examples of cosmetic products or rather their formulations are given in Tables 5 to 8.

Cosmetic and/or Dermatological Preparations

The preparations according to the invention may be used for the production of cosmetic and/or dermatological preparations such as, for example, foam baths, shower baths, creams, gels, lotions, alcohol and water/alcohol solutions, emulsions, wax/fat compounds, stick preparations, powders or ointments. These preparations may additionally contain mild surfactants, oil components, emulsifiers, pearlizing waxes, consistency factors, thickeners, superfatting agents, stabilizers, polymers, silicone compounds, fats, waxes, lecithins, phospholipids, biogenic agents, antioxidants, deodorants, antiperspirants, film formers, swelling agents, insect repellents, hydrotropes, solubilizers, preservatives, perfume oils, dyes and the like as further auxiliaries and additives.

Surfactants

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols, esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols (cf. DE 19756377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di-and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates such as, for example, Dicaprylyl Carbonate (Cetiol® CC), Guerbet carbonates based on C C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Dicaprylyl Ether (Cetiol® OE), ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Emulsifiers

Suitable emulsifiers are, for example, nonionic surfactants from at least one of the following groups:

products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alkyl group and ethoxylated analogs thereof;

addition products of 1 to 15 mol ethylene oxide onto castor oil and/or hydrogenated castor oil;

addition products of 15 to 60 mol ethylene oxide onto castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 mol ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 mol ethylene oxide;

mixed esters of pentaerythritol, fatty acids, citric acid and fatty alcohol according to DE 1165574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethyleneglycol-30 Dipolyhydroxystearate;

polymer emulsifiers, for example Pemulen types (TR-1, TR-2) of Goodrich;

polyalkylene glycols and

glycerol carbonate.

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as refatting agents for cosmetic formulations from DE 2024051 PS.

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide with the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide with the sorbitan esters mentioned are also suitable.

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethylimidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C₈₋₁₈ alkyl or acyl group, contain at least one free amino group and at least one —COOH— or —SO₃H-group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinylimidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grunau), quaternized wheat poly-peptides, polyethyleneimine, cationic silicone polymers such as, for example, Amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosm. Toil. 108, 95 (1993).

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Antioxidants

Besides primary sun protection factors, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Biogenic Agents

Biogenic agents in the context of the invention are, for example, tocopherol, tocopherol acetate, tocopherol palmitate, ascorbic acid, deoxyribonucleic acid and fragmentation products thereof, retinol, bisabolol, allantoin, phytantriol, panthenol, AHA acids, amino acids, ceramides, pseudoceramides, essential oils, plant extracts and additional vitamin complexes.

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers. Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of labdanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

non-aqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH regulators, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Insect Repellents

Suitable insect repellents are N,N-diethyl-m-toluamide, pentane-1,2-diol or Ethyl Butylacetylaminopropionate.

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Perfume Oils

Suitable perfume oils are mixtures of natural and synthetic fragrances. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetivert oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, a-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Dyes

Suitable dyes are any of the substances suitable and approved for cosmetic purposes as listed, for example, in the publication “Kosmetische Färbemittel” of the Farbstoffkommission der Deutschen Forschungsgemeinschaft, Verlag Chemie, Weinheim, 1984, pages 81 to 106. These dyes are normally used in concentrations of 0.001 to 0.1% by weight, based on the mixture as a whole.

### EXAMPLES

### Example 1

### Extraction of the Fungi With Aqueous Ethanol

300 g dried Grifola frondosa fungi were introduced into a glass reactor containing 3 liters 96% by weight aqueous ethanol. The infusion was extracted with stirring for 1 hour at boiling temperature. The mixture was then cooled to 20° C. and the supernatant colloidal liquid was separated from the residue by filtration through a depth filter with a mean porosity of 450 nm (from Seitz, Bordeaux/France). The alcohol was then removed under reduced pressure at 45° C. and the residue was dried at 50° C. The yield of dry product was 10.9% by weight, based on the dry weight of fungi used.

### Example 2

### Extraction of the Fungi With Aqueous Ethanol and Water

Example 1 was repeated except that 100 g of the dry product were then introduced into a glass reactor containing 1 liter of an aqueous solution with a pH of 7.5. The pH was adjusted with a 4 N NaOH solution. Extraction was carried out with stirring for 1 h at room temperature and the extract was centrifuged for 15 mins. at a speed of 5,000 G. The supernatant liquid was separated from the residue by filtration. Filtration was carried out as described in Example 1 and the residue was spray dried. The yield of dry product was 20.3% by weight, based on the dry weight of fungi used. The percentage content of proteins in the dry product was 9.2% by weight and the percentage content of sugars therein was 53.8% by weight.

### Example 3

### Extraction of the Fungi With Hexane

Example 1 was repeated except that the extraction of 500 g of dried Grifola frondosa was carried out with 5 liters of hexane. The extraction process was carried out under reflux with stirring for 1 h at boiling temperature and the extract was further processed as described. Filtration was carried out as described in Example 1. The solvent was then removed under reduced pressure and the residue was dried at 50° C. The yield of dry product was 1.9% by weight, based on the dry weight of fungi used.

### Example 4

### Analysis

The extracts of Examples 1 to 3 were analyzed on an HPLC cation exchanger in a Chromopack CP 28350 (300×6.5) column at a temperature of 70° C. and a flow rate of 0.8 ml/min. and using a mobile phase of a 0.01 N H₂SO₄ solution and isocratic elution. The detector was a UV detector with a wavelength of 210 nm. The fumaric acid content of the individual extracts was to be investigated. The retention time of a standard of fumaric acid was 9.29 mins. Quantities of 10 μl of a 5% by weight extract were injected.

TABLE 1
Analytical determination of the fumaric acid
content of the extracts of Examples 1-3

| | Fumaric |
|---|---|
| Extract | acid content (g/100 g extract) |
| Extract of Example 3/5% by weight | 0 |
| Extract of Example 1/5% by weight | 0.0025 |
| Extract of Example 2/5% by weight | 0.038 |

The results set out in the Table show that no fumaric acid can be detected in the extract of Example 3 and that the extracts of Examples 1 and 2 contain only very small quantities of fumaric acid.

### Example 5

### Skin Regenerating and Revitalizing Activity

The object of this test is to demonstrate the regenerating and revitalizing activity of extracts of Grifola frondosa on human fibroblast cultures in vitro.

Method 1: Effects on Cell Growth

Human fibroblasts were inoculated with 10% by weight foetal calf serum in a defined nutrient medium (DMEM=Dulbecco Minimum Essential Medium, a product of Life Technologie S.a.r.l.) and incubated for 24 h at 37° C. in a 5% CO₂ atmosphere. The nutrient medium containing foetal calf serum was then replaced by a nutrient medium of DMEM without foetal calf serum. Active substance in the form of the Grifola extracts of Examples 1 and 2 was then added to this nutrient medium in various concentrations. For comparison, a test series of human fibroblasts with no active substance was incubated as control. After the fibroblasts had been incubated for 3 days in the nutrient medium, growth and metabolic activity were evaluated by counting the cells with a particle counter and determining the intracellular ATP content by Vasseur's method (Journal Français Hydrologie, 1981, 9, 149-156), the cell protein content by Bradford's method (Anal. Biochem. 1976, 72, 248-254) and the cellular DNA content by Desaulnier's method (In vitro, 1998, 12(4), 409-422). With concentrations of 0.001 to 0.1% by weight of fungal extract of Examples 1 and 2, an increase in the percentage ATP content of 8 to 23% was obtained by comparison with the control. The percentage cell protein content was increased by 5 to 32% for concentrations of 0.001 to 0.1% by weight of fungal extract of Examples 1 and 2. The percentage cellular DNA content was determined after 3 and 6 days' incubation at concentrations of 0.001 to 0.003% by weight fungal extract of Example 1. It was found that the percentage content had increased by 10 to 42% by comparison with the control.

TABLE 2
Increase in the protein content and ATP content
in human fibroblasts after incubation with the
extracts of Examples 1 to 3 in comparison to
incubation with no fungal extract

| | Concentration | Protein content in | |
|---|---|---|---|
| Substance | % by weight | % | ATP content |
| Control | | 100 | 100 |
| Extract of Example 1 | 0.001 | 114 | 108 |
| | 0.003 | 132 | 123 |
| | 0.01 | 126 | 81 |
| Extract of Example 2 | 0.001 | 115 | 113 |
| | 0.003 | 105 | 118 |
| | 0.01 | 98 | 116 |
| | 0.03 | 88 | 121 |
| Extract of Example 3 | 0.0001 | 108 | 104 |
| | 0.0003 | 119 | 100 |
| | 0.001 | 136 | 108 |
| | 0.003 | 129 | 86 |

TABLE 3
Increase in the DNA content in human
fibroblasts after incubation with the
extract of Example 1 in comparison
to incubation with no fungal extract

| | Concentration | 3 days' | 6 days' |
|---|---|---|---|
| Substance | % by weight | incubation in % | incubation in % |
| Control | | 100 | 100 |
| Extract of Example 1 | 0.001 | 128 | 138 |
| | 0.003 | 110 | 142 |

The study shows that the Grifola frondosa extracts of Examples 1 to 3 stimulate the growth and metabolism of human fibroblasts in vitro to a considerable extent.

Method 2: Improvement of Viability

The test was carried out on human fibroblasts. It enables a certain number of parameters to be quantitatively determined on the resting cells. The cultivation of the cells corresponds to the cultivation of method 1 except for the incubation time. The incubation time for this test was 72 h. Viability was evaluated by calorimetric determination of the percentage protein content by Bradford's method (Anal. Biochem. 1976, 72, 248-254). The test was carried out three times and then repeated twice so that there were six results per plant extract and hence per batch which were all averaged. The results were expressed in % by comparison with the control.

TABLE 4
Increase in the protein content in human
fibroblasts after incubation with
extracts of Examples 1 to 3 in comparison
to incubation with no fungal extract

| | Concentration | |
|---|---|---|
| Substance | % by weight | Protein content in % |
| Control | | 100 |
| Extract of Example 1 | 0.001 | 121 |
| | 0.003 | 135 |
| | 0.01 | 180 |
| Extract of Example 2 | 0.001 | 115 |
| | 0.003 | 111 |
| | 0.01 | 126 |
| Extract of Example 3 | 0.001 | 105 |
| | 0.003 | 112 |
| | 0.01 | 125 |

The Grifola frondosa extracts of Examples 1 to 3 in concentrations of 0.001 to 0.01% by weight increase the content of proteins in human fibroblasts in comparison with the control by 5 to 80% in in vitro tests. These results show that the Grifola frondosa extracts have a high capacity for stimulating the metabolism of fibroblasts. The extracts show regenerating and revitalizing activity of human fibroblasts and, accordingly, may be used as energy sources and as anti-ageing agents in cosmetic and dermatological preparations.

6. Anti-Inflammatory Activity

Background: Cutaneous inflammation can be produced by UV-B radiation through the stimulation of epidermal keratinocytes. This is followed by the onset of acute leucocyte infiltration.

This activation of the leucocytes, especially neutrophilic granulocytes, is known as “respiratory burst” and can lead to tissue destruction by reactive oxygen radicals released (reactive oxygen species—ROS) and by lyosomal enzymes.

Method: Anti-inflammatory activity was studied on a cell line of human leucocytes (neutrophilic granulocytes). To this end, the cells were incubated with various concentrations of extracts of Examples 1 and 3 to be tested, after which a respiratory burst was induced by yeast cell extract (“zymosan”). The content of oxygen radicals intermediately formed and “dumped” oxygen radicals was determined by reaction with luminol via the luminescence and quantitatively evaluated. The luminescence yield falls in the presence of substances with radical-trapping properties. The results are set out in Table 4. The percentage of released radicals is shown in %-absolute (“luminol test”). For control purposes, the number of intact cells was determined with a particle counter and is shown in % by comparison with the control.

TABLE 5
Determination of the released oxygen radicals

| | Concentration | ROS content | |
|---|---|---|---|
| Substance | % by weight | (%/control) | No. of intact cells |
| Control | | 100 | 100 |
| Extract of Example 1 | 0.001 | 160 | 101 |
| | 0.01 | 187 | 104 |
| | 0.1 | 68 | 101 |
| Extract of Example 3 | 0.001 | 51 | 100 |

It can be seen from the above results that the extracts of Example 1 according to the invention have a strong anti-inflammatory effect in a concentration of 0.1%. The extracts of Example 3 according to the invention have a strong anti-inflammatory effect in a concentration of only 0.001%. The effects on human leucocytes were obtained without any toxicological effect on the cells. In conjunction with the results of Example 4, it can be seen that the extracts with the strong anti-inflammatory effects contain little or no fumaric acid. Accordingly, the anti-inflammatory effect cannot be attributed to the presence of fumaric acid.

### Example 7

### Effectiveness in Protecting Cells Against UV-B in Human Keratinocytes Cultivated in vitro

Background: UV-B rays cause inflammation (erythema, edema) by activating an enzyme, namely phospholipase A2 or PLA2, which removes arachidonic acid from the phospholipids of the plasma membranes. Arachidonic acid is the precursor of the prostaglandins which cause inflammation and cell membrane damage; the prostaglandins E2 (=PGE2) are formed by cyclooxygenase.

Method: The effect of UV-B radiation was investigated in vitro in keratinocytes by determining the release of the cytoplasm enzyme LDH (lactate dehydrogenase). This enzyme serves as a marker for cell damage.

To carry out the tests, a defined medium containing foetal calf serum was inoculated with the keratinocytes and the fungal extract (diluted with saline solution) was added 72 hours after the inoculation.

The keratinocytes were then exposed to a dose of UV-B (50 mJ/cm²—tubes: DUKE FL40E).

After incubation for another day at 37° C./5% CO₂, the LDH content in the supernatant was determined. The LDH (lactate dehydrogenase) content was determined by an enzyme reaction (kit used to determine LDH levels from Roche). The number of adhering keratinocytes was determined (after trypsin treatment) with a particle counter.

TABLE 6
Cell protecting effect of Grifola frondosa
extracts against UVB rays; results in %
based on the control, average value from
2 tests each repeated twice

| | | Content of |
|---|---|---|
| | Number of keratinocytes | LDH released |
| Control without UV | 100 | 0 |
| Control with UV-B (50 | 19 | 100 |
| mJ/cm²) | | |
| UV-B + extract of Example 1/ | 23 | 74 |
| 0.003% by weight | | |
| UV-B + extract of Example 2/ | 35 | 49 |
| 0.03% by weight | | |
| UV-B + Aspirin ®/0.03% by | | |
| weight (SIGMA) | | |

The results of these tests show that an extract of Grifola frondosa according to the invention reduces the effect of UV-B radiation on the number of keratinocytes and on the content of LDH released. Accordingly, the described extracts have the ability to reduce cell membrane damage caused by UV-B radiation.

The effects and positive activities of the Grifola frondosa extracts therefore contain a very strong

activating (stimulating), revitalizing and regenerating activity on the metabolism,

strong anti-inflammatory activity,

trapping and neutralizing of radicals and reactive oxygen species,

activity in reducing cell membrane damage caused by UV-B radiation.

### Example 8

### Exemplary Formulations of Cosmetic Preparations Containing Grifola frondosa Extracts

The Grifola frondosa extracts obtained in accordance with Examples 1 to 3 were used in the following formulations K1 to K21 and 1 to 13 according to the invention. The cosmetic preparations thus produced showed very good skin-care properties in relation to comparison formulations C1, C2 and C3 coupled with good dermatological compatibility. The preparations according to the invention are also stable to oxidative decomposition.

TABLE 7
Soft cream formulations K1 to K7
(All quantities in % by weight,
based on the cosmetic preparation)

| | INCI name | | | | | | | |
|---|---|---|---|---|---|---|---|---|
| | K1 | K2 | K3 | K4 | K5 | K6 | K7 | C1 |
| Glyceryl Stearate (and) | 8.0 | 8.0 | 8.0 | 8.0 | 8.0 | 8.0 | 8.0 | 8.0 |
| Ceteareth-12/20 (and) | | | | | | | | |
| Cetearyl Alcohol (and) Cetyl | | | | | | | | |
| Palmitate | | | | | | | | |
| Cetearyl Alcohol | 2.0 | 2.0 | 2.0 | 2.0 | 2.0 | 2.0 | 2.0 | 2.0 |
| Dicaprylyl Ether | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 3.0 | 3.0 | 3.0 | 3.0 | 3.0 | 3.0 | 3.0 | 3.0 |
| Glycerin (86% by weight) | 3.0 | 3.0 | 3.0 | 3.0 | 3.0 | 3.0 | 3.0 | 3.0 |
| Extract of Examples 1-3 | 0.5 | 0.5 | 0.5 | 0.5 | 0.5 | 0.5 | 0.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

TABLE 8
Night cream formulations K8 to K14
(All quantities in % by weight, based on the cosmetic preparation)

| | INCI name | | | | | | | |
|---|---|---|---|---|---|---|---|---|
| | K8 | K9 | K10 | K11 | K12 | K13 | K14 | C2 |
| Polyglyceryl-2 Dipolyhydroxystearate | 4.0 | 4.0 | 4.0 | 4.0 | 4.0 | 4.0 | 4.0 | 5.0 |
| Polyglyceryl-3 Diisostearate | 2.0 | 2.0 | 2.0 | 2.0 | 2.0 | 2.0 | 2.0 | 2.0 |
| Cera Alba | 2.0 | 2.0 | 2.0 | 2.0 | 2.0 | 2.0 | 2.0 | 2.0 |
| Zinc Stearate | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 8.0 | 8.0 | 8.0 | 8.0 | 8.0 | 8.0 | 8.0 | 8.0 |
| Dicaprylyl Ether | 5.0 | 5.0 | 5.0 | 5.0 | 5.0 | 5.0 | 5.0 | 5.0 |
| Magnesium sulfate | 1.0 | 1.0 | 1.0 | 1.0 | 1.0 | 1.0 | 1.0 | 1.0 |
| Glycerin (86% by weight) | 5.0 | 5.0 | 5.0 | 5.0 | 5.0 | 5.0 | 5.0 | 5.0 |
| Extract of Examples 1-3 | 0.5 | 0.5 | 0.5 | 0.5 | 0.5 | 0.5 | 0.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

TABLE 9
W/O body lotion formulations K15 to K21.
(All quantities in % by weight, based on the cosmetic preparation)

| | INCI name | | | | | | | |
|---|---|---|---|---|---|---|---|---|
| | K15 | K16 | K17 | K18 | K19 | K20 | K21 | C3 |
| PEG-7 Hydrogenated Castor Oil | 7.0 | 7.0 | 7.0 | 7.0 | 7.0 | 7.0 | 7.0 | 7.0 |
| Decyl Oleate | 7.0 | 7.0 | 7.0 | 7.0 | 7.0 | 7.0 | 7.0 | 7.0 |
| Cetearyl Isononanoate | 7.0 | 7.0 | 7.0 | 7.0 | 7.0 | 7.0 | 7.0 | 7.0 |
| Glycerin (86% by weight) | 5.0 | 5.0 | 5.0 | 5.0 | 5.0 | 5.0 | 5.0 | 5.0 |
| MgSO₄·7H₂O | 1.0 | 1.0 | 1.0 | 1.0 | 1.0 | 1.0 | 1.0 | 1.0 |
| Extract of Examples 1-3 | 1.5 | 1.5 | 1.5 | 1.5 | 1.5 | 1.5 | 1.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

¹⁾Deoxyribonucleic acid: molecular weight ca. 70,000, purity (determined by spectrophotometric measurement of absorption at 260 nm and 280 nm): at least 1.7

TABLE 10
Formulations
Cosmetic preparations conditioner
(water, preservative to 100% by weight)

| | Composition (INCI) | | | |
|---|---|---|---|---|
| | 1 | 2 | 3 | 4 |
| Texapon ® NSO | 38.0 | 38.0 | 25.0 | — |
| Sodium Laureth Sulfate | | | | |
| Texapon ® SB 3 | — | — | 10.0 | — |
| Disodium Laureth Sulfosuccinate | | | | |
| Plantacare ® 818 | 7.0 | 7.0 | 6.0 | — |
| Coco Glucosides | | | | |
| Plantacare ® PS 10 | — | — | — | 20.0 |
| Sodium Laureth Sulfate (and) Coco | | | | |
| Glucosides | | | | |
| Dehyton ® PK 45 | — | — | 10.0 | — |
| Cocamidopropyl Betaine | | | | |
| Lamesoft ® PO 65 | 3.0 | | | 4.0 |
| Coco-Glucoside (and) Glyceryl Oleate | | | | |
| Lamesoft ® LMG | — | 5.0 | — | — |
| Glyceryl Laurate (and) Potassium Cocoyl | | | | |
| Hydrolyzed Collagen | | | | |
| Euperlan ® PK 3000 AM | — | 3.0 | 5.0 | 5.0 |
| Glycol Distearate (and) Laureth-4 (and) | | | | |
| Cocamidopropyl Betaine | | | | |
| Extract of Examples 1 to 3 | 1.0 | 1.0 | 1.0 | 1.0 |
| Arlypon ® F | 3.0 | 3.0 | 1.0 | — |
| Laureth-2 | | | | |
| Sodium Chloride | — | 1.5 | — | 1.5 |

Cosmetic preparations “2-in-1” shower
bath (water, preservative to 100% by weight)

| | Composition (INCI) | | | |
|---|---|---|---|---|
| | 5 | 6 | 7 | 8 |
| Texapon ® NSO | 30.0 | 25.0 | | 25.0 |
| Sodium Laureth Sulfate | | | | |
| Plantacare ® 818 | | | | 8.0 |
| Coco Glucosides | | | | |
| Plantacare ® 2000 | | 8.0 | | |
| Decyl Glucoside | | | | |
| Plantacare ® PS 10 | | | 20.0 | |
| Sodium Laureth Sulfate (and) Coco | | | | |
| Glucosides | | | | |
| Dehyton ® PK 45 | | 10.0 | 10.0 | |
| Cocamidopropyl Betaine | | | | |
| Lamesoft ® PO 65 | 5.0 | | | |
| Coco-Glucoside (and) Glyceryl Oleate | | | | |
| Lamesoft ® LMG | | 5.0 | 5.0 | |
| Glyceryl Laurate (and) Potassium Cocoyl | | | | |
| Hydrolyzed | | | | |
| Collagen | | | | |
| Gluadin ® WQ | 3.0 | | | |
| Laurdimonium Hydroxypropyl Hydrolyzed | | | | |
| Wheat Protein | | | | |
| Gluadin ® WK | | | | |
| Sodium Cocoyl Hydrolyzed Wheat Protein | | | | |
| Euperlan ® PK 3000 AM | 5.0 | 3.0 | 4.0 | — |
| Glycol Distearate (and) Laureth-4 (and) | | | | |
| Cocamidopropyl Betaine | | | | |
| Panthenol | 0.5 | — | — | 0.5 |
| Extract of Examples 1 to 3 | 1.0 | 1.0 | 1.0 | 1.0 |
| Arlypon ® F | 2.6 | 1.6 | — | 1.0 |
| Laureth-2 | | | | |
| Sodium Chloride | — | — | — | — |

Cosmetic preparations foam bath
(water, preservative to 100% by weight)

| | Composition (INCI) | | | | |
|---|---|---|---|---|---|
| | 9 | 10 | 11 | 12 | 13 |
| Texapon ® NSO | — | 30.0 | 30.0 | — | 25.0 |
| Sodium Laureth Sulfate | | | | | |
| Plantacare ® 818 | — | 10.0 | — | — | 20.0 |
| Coco Glucosides | | | | | |
| Plantacare ® PS 10 | 22.0 | — | 5.0 | 22.0 | — |
| Sodium Laureth Sulfate (and) Coco | | | | | |
| Glucosides | | | | | |
| Dehyton ® PK 45 | 15.0 | 10.0 | 15.0 | 15.0 | 15.0 |
| Cocamidopropyl Betaine | | | | | |
| Monomuls ® 90-O 18 | 0.5 | | | | |
| Glyceryl Oleate | | | | | |
| Lamesoft ® PO 65 | | 3.0 | | 3.0 | |
| Coco-Glucoside (and) Glyceryl | | | | | |
| Oleate | | | | | |
| Cetiol ® HE | | | 2.0 | | 2.0 |
| PEG-7 Glyceryl Cocoate | | | | | |
| Nutrilan ® I-50 | 5.0 | | | | |
| Hydrolyzed Collagen | | | | | |
| Gluadin ® W 40 | | 5.0 | | 5.0 | |
| Hydrolyzed Wheat Gluten | | | | | |
| Gluadin ® WK | | | | 7.0 | |
| Sodium Cocoyl Hydrolyzed Wheat | | | | | |
| Protein | | | | | |
| Euperlan ® PK 3000 AM | 5.0 | — | — | 5.0 | — |
| Glycol Distearate (and) Laureth-4 | | | | | |
| (and) Cocamidopropyl Betaine | | | | | |
| Arlypon ® F | | | 1.0 | | |
| Laureth-2 | | | | | |
| Sodium Chloride | 1.0 | | 1.0 | | 2.0 |
| Extract of Examples 1 to 3 | 1.0 | 1.0 | 1.0 | 1.0 | 1.0 |

(1-2) shower bath,
(3) shower gel,
(4) wash lotion

1. The use of extracts of the fungus Grifola frondosa in cosmetic and/or dermatological skin care preparations.

2. The use of extracts of the fungus Grifola frondosa in protective and restorative care preparations for stimulating the metabolism of the skin cells with skin revitalizing and reactivating activity.

3. The use of extracts of the fungus Grifola frondosa in care preparations for stimulating the immune defense of the skin.

4. The use of extracts of the fungus Grifola frondosa in cosmetic and/or dermatological care preparations for stimulating the synthesis of dermal macromolecules selected from the group consisting of collagen, elastin, fibronectin, proteoglycans and hyaluronic acid and salts thereof.

5. The use of extracts of the fungus Grifola frondosa in cosmetic and/or dermatological care preparations for the preventive or healing treatment of signs of skin ageing.

6. The use claimed in claim 5, characterized in that UV-induced skin ageing is treated.

7. The use of extracts of the fungus Grifola frondosa as a cosmetic and/or dermatological anti-inflammatory care preparation.

8. The use claimed in claim 8, characterized in that inflammation of the skin caused by UV radiation or contamination of the skin is treated.

9. The use claimed in claim 8, characterized in that bacterially and hormonally induced changes to the skin, for example acne, are treated.

10. The use of extracts of the fungus Grifola frondosa in cosmetic and/or dermatological care preparations for sensitive skin.

11. The use of extracts of the fungus Grifola frondosa in cosmetic and/or dermatological care preparations for improving wound healing.

12. The use of extracts of the fungus Grifola frondosa in cosmetic and/or dermatological sun protection compositions.
